(19) United States
(12) Patent Application Publication
(10) Pub. No.: US 2026/0198858 A1
(43) Pub. Date: Jul. 16, 2026

(54) FINGER-MOUNTED DEVICE

(71) Applicant: Murata Manufacturing Co., Ltd., Nagaokakyo-shi (JP)

(72) Inventor: Toru SHIMUTA, Nagaokakyo-shi (JP)

(21) Appl. No.: 19/564,530

(22) Filed: Mar. 12, 2026

Related U.S. Application Data
(63) Continuation of application No. PCT/JP2024/023779, filed on Jul. 1, 2024.

(30) Foreign Application Priority Data
Oct. 13, 2023 (JP) 2023-177474

(51) Int. Cl. A61B 5/00; A61B 5/01

(52) U.S. Cl. CPC A61B 5/6826; A61B 5/0059; A61B 5/01; A61B 2560/0214; A61B 2560/0223; A61B 2560/0462; A61B 2562/0219; A61B 2562/0271

(57) ABSTRACT

A first light source and a second light source held by an annular structure emit light having respective wavelengths. A light receiver held by the annular structure receives reflected or scattered light associated with the first light source and the second light source. A control unit controls the first light source and the second light to emit intermittently and determines whether the annular structure is mounted on a finger, based on received light levels by the light receiver. A distance from the light receiver to the second light source is longer than a distance from the light receiver to the first light source. The control unit is configured to perform a mounted-state determination process that determines whether the annular structure is mounted on the finger based on the received light levels by the light receiver, and to perform a biometric-information measurement process based on the received light levels.

### CROSS REFERENCE TO RELATED APPLICATIONS

This application is a continuation of International Application No. PCT/JP 2024/023779, filed Jul. 1, 2024, which claims priority to Japanese Patent Application No. 2023-177474, filed Oct. 13, 2023, the contents of each of which are hereby incorporated by reference in their entirety.

### TECHNICAL FIELD

The present disclosure relates to a finger-mounted device.

### BACKGROUND

A related example of a biometric information sensor includes a light-emitting portion and a light-receiving portion, such as that described in Japanese Unexamined Patent Application Publication No. 2018-161250. The biometric information sensor disclosed therein is mounted on a wrist and has a function of determining whether the biometric information sensor is properly mounted. Processing for determining this includes a proximity determination process in which, when light is emitted from the light-emitting portion, whether reflected light is detected by the light-receiving portion is determined. Reflected light from the wrist is detected by the light-receiving portion with the biometric information sensor mounted on the wrist. In the case where the biometric information sensor is not mounted on the wrist, reflected light does not reach the light-receiving portion. For this reason, whether the biometric information sensor is properly mounted on the wrist can be determined from the result of detection made by the light-receiving portion.

As for a finger-mounted device, a light-emitting portion and a light-receiving portion are disposed on an inner circumferential surface of an annular structure that is mounted on a finger. A part of light that is emitted from the light-emitting portion is reflected by the inner circumferential surface of the annular structure and is received by the light-receiving portion. Even when the device is not mounted on the finger, a part of the light is incident on the light-receiving portion, and accordingly, a method of determining whether the device is mounted on the finger depending on the presence or absence of light received by the light-receiving portion reduces the accuracy of determination.

### SUMMARY OF THE INVENTION

Some exemplary aspects of the present disclosure provide a finger-mounted device that improves the accuracy of determination of whether the finger-mounted device is mounted on a finger.

According to an exemplary aspect of the present disclosure, a finger-mounted device is provided that includes an annular structure configured to be mountable on a finger; a first light source and a second light source that are held by the annular structure and that emit light having respective wavelengths different from each other into a space that is surrounded by the annular structure; a light receiver that is held by the annular structure and configured to receive light including reflected or scattered light associated with the first light source and the second light source; and a control unit that is configured to control the first light source and the second light source to emit light intermittently and to determine whether the annular structure is mounted on the finger, based on a received light level that is measured by the light receiver. A distance from the light receiver to the second light source is longer than a distance from the light receiver to the first light source. The control unit is configured to perform a mounted-state determination process that determines that the annular structure is mounted on the finger when a first received light level of the light receiver associated with the first light source exceeds a first threshold, and a second received light level of the light receiver associated with the second light source exceeds a second threshold. The control unit is also configured to perform a biometric-information measurement process of acquiring biometric information, based on at least the first received light level or the second received light level.

A mounted state is determined by using a first received light level when a first light source emits light and a second received light level when a second light source emits light, and consequently, the accuracy of determination is improved.

### BRIEF DESCRIPTION OF DRAWINGS

FIG. 1A schematically illustrates a perspective view of a finger-mounted device according to a first exemplary embodiment, and FIG. 1B is a sectional view of the finger-mounted device according to the first exemplary embodiment taken along a direction perpendicular to an insertion/removal direction.

FIG. 2 illustrates a block diagram for describing the function of the finger-mounted device according to the first exemplary embodiment.

FIG. 3A and FIG. 3B are graphs illustrating a change in a received light level that is measured by a light receiver over time.

FIG. 4A and FIG. 4B are graphs illustrating the change in the received light level that is measured by the light receiver over time.

FIG. 5A and FIG. 5B are graphs illustrating the change in the received light level that is measured by the light receiver over time.

FIG. 6 is a flowchart illustrating a procedure for a mounted-state determination process that is performed by a control unit of the finger-mounted device according to the first exemplary embodiment while biometric information is not measured.

FIG. 7 is a flowchart illustrating a procedure for the mounted-state determination process that is performed by the control unit of the finger-mounted device according to the first exemplary embodiment while the biometric information is measured.

FIG. 8 illustrates a sectional view of a finger-mounted device according to a second exemplary embodiment.

FIG. 9 illustrates a block diagram for describing the function of the finger-mounted device according to the second exemplary embodiment.

FIG. 10 is a flowchart illustrating a procedure for a mounted-state determination process that is performed by a control unit of the finger-mounted device according to the second exemplary embodiment while the biometric information is not measured.

FIG. 11 schematically illustrates a sectional view of a finger-mounted device according to a third exemplary embodiment.

FIG. 12 illustrates a block diagram for describing the function of the finger-mounted device according to the third exemplary embodiment.

FIG. 13 schematically illustrates an inner circumferential surface of a finger-mounted device according to a fourth exemplary embodiment when viewed in the insertion/removal direction of a finger.

FIG. 14 illustrates a sectional view of a finger-mounted device according to a fifth exemplary embodiment with the finger-mounted device mounted on a finger.

FIG. 15 illustrates a block diagram of a finger-mounted device according to a sixth exemplary embodiment and an external device related to the operation of the finger-mounted device.

FIG. 16 is a flowchart illustrating a procedure for a mounted-state determination process that is performed by a control unit of a finger-mounted device according to a seventh exemplary embodiment while the biometric information is not measured.

FIG. 17 schematically illustrates a sectional view of a finger-mounted device according to an eighth exemplary embodiment.

FIG. 18 is a graph illustrating changes in temperatures that are measured by a first temperature sensor and a second temperature sensor of the finger-mounted device according to the eighth exemplary embodiment and the standard deviations of the temperatures within a predetermined period of time.

FIG. 19 is a flowchart illustrating a procedure for a mounted-state determination process that is performed by a control unit of the finger-mounted device according to the eighth exemplary embodiment while the biometric information is measured.

### DETAILED DESCRIPTION OF EMBODIMENTS

First Exemplary Embodiment

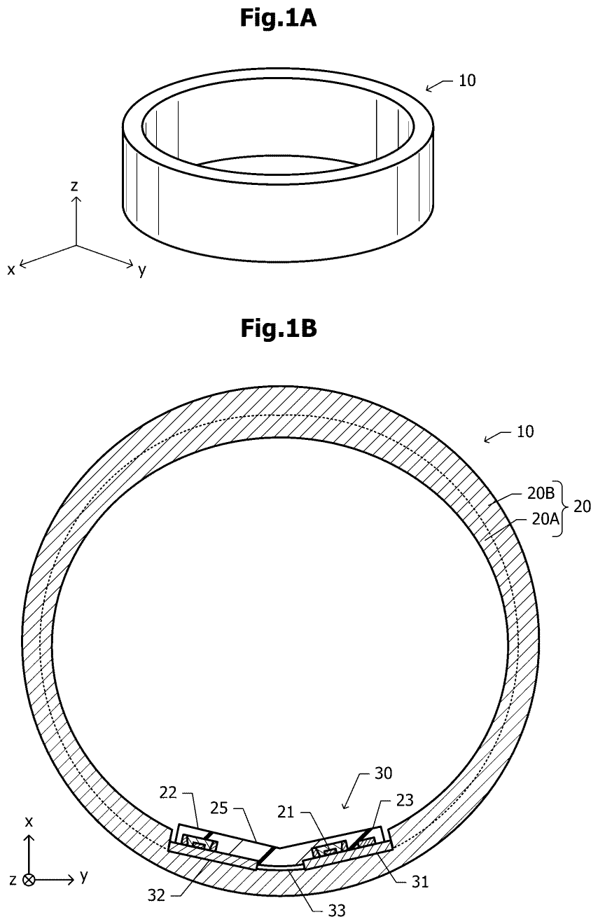

A finger-mounted device according to a first exemplary embodiment will be described with reference to FIG. 1A to FIG. 7.

FIG. 1A schematically illustrates a perspective view of a finger-mounted device 10 according to the first exemplary embodiment. The finger-mounted device 10 has an annular shape. A finger is inserted into a hollow portion of the finger-mounted device 10 that has an annular shape, and consequently, the finger-mounted device 10 is mounted on the finger. The finger-mounted device 10 that is mounted on the finger acquires biometric information such as a photoplethysmogram. An xyz rectangular coordinate system is defined such that the insertion/removal direction of the finger when the finger-mounted device 10 is mounted on the finger is a z-direction.

FIG. 1B is a sectional view of the finger-mounted device 10 according to the first exemplary embodiment taken along a direction perpendicular to a z-axis. The finger-mounted device 10 includes an annular structure 20 that extends along an outer circumferential line of a circle or an ellipse and a sensor module 30 that is held by the annular structure 20. The annular structure 20 includes an inner member 20A that has an annular shape and an outer member 20B that surrounds an outer circumferential surface of the inner member 20A.

The inner member 20A has an opening portion that extends from an inner circumferential surface thereof to the outer circumferential surface. The sensor module 30 is fitted into the opening portion. The sensor module 30 is fixed to the inner member 20A by using an adhesive or an adhesive tape.

The sensor module 30 includes two rigid substrates 31 and 32 and a flexible substrate 33 that connects these. An example of the two rigid substrates 31 and 32 and the flexible substrate 33 is a rigid-flexible substrate that includes a rigid substrate and a flexible substrate integrated with each other. The two rigid substrates 31 and 32 are disposed at different positions in a circumferential direction.

A first light source 21 and a light receiver 23 are mounted at different positions in the circumferential direction on an inner surface (a surface that faces a space that is surrounded by the annular structure 20) of the rigid substrate 31. A second light source 22 is mounted on an inner surface of the rigid substrate 32. The light receiver 23, the first light source 21, and the second light source 22 are located in this order in the circumferential direction. A distance from the light receiver 23 to the second light source 22 is longer than a distance from the light receiver 23 to the first light source 21.

For example, the distance from the light receiver 23 to the first light source 21 is no less than 1 mm and no more than 3 mm. For example, the distance from the light receiver 23 to the second light source 22 is no less than 5 mm and no more than 20 mm. For example, the center point of an active region such as a light-emitting region or a light-receiving region can be used as a reference point for identifying relative positions of the first light source 21, the second light source 22, and the light receiver 23. A resin member 25 is disposed so as to cover inner surfaces of the first light source 21, the second light source 22, the light receiver 23, the rigid substrates 31 and 32, and the flexible substrate 33.

The first light source 21 and the second light source 22 emit light in respective wavelength ranges different from each other toward the space that is surrounded by the annular structure 20. The first light source 21 and the second light source 22 emit light toward the finger with the finger-mounted device 10 mounted on the finger. Examples of the first light source 21 and the second light source 22 include a vertical-cavity surface-emitting laser (VCSEL) and a light-emitting diode (LED). Examples of the light receiver 23 include a photodiode and a phototransistor. Resin such as epoxy, silicone, acrylic, polycarbonate, urethane, polyethylene terephthalate, polypropylene, PET, or ABS resin is used for the resin member 25.

Parts of the light emitted from the first light source 21 and the second light source 22 are reflected by the inner circumferential surface of the annular structure 20 with the finger-mounted device 10 unmounted from the finger and are incident on the light receiver 23. The light emitted from the first light source 21 and the second light source 22 scatters in the finger (in vivo) with the finger-mounted device 10 mounted on the finger, and scattered light is incident on the light receiver 23.

For example, the first light source 21 emits the light in a wavelength range from blue to yellow-green, and the second light source 22 emits the light in a wavelength range from red to near infrared. The light in the wavelength range from red to near infrared is less absorbed by a living body than the light in the wavelength range from blue to yellow-green and accordingly deeply enters the living body. For this reason, biometric information about a relatively shallow region is acquired from the result of measurement of a received light level (referred to below as a first received light level) when the first light source 21 operates, and biometric information about a relatively deep region is acquired from the result of measurement of a received light level (referred to below as a second received light level) when the second light source 22 operates.

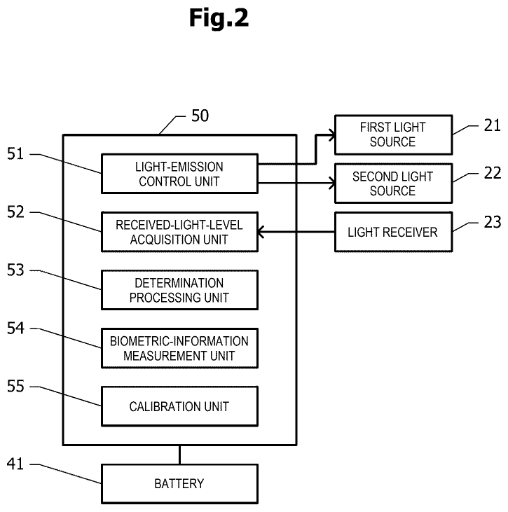

FIG. 2 illustrates a block diagram for describing the function of the finger-mounted device 10 according to the first exemplary embodiment.

The finger-mounted device 10 according to the first exemplary embodiment includes a control unit 50. For example, the control unit 50 includes one or multiple integrated circuits and is held by the annular structure 20. For example, the control unit 50 is mounted on, for example, the rigid substrate 31 or 32. In an exemplary aspect, the one or multiple integrated circuits can be configured to execute an application (e.g., software) stored on electronic memory of the finger-mounted device 10 to perform the methods, algorithms and calculations described herein. This execution can be performed through, for example, one or more programmable components, such as a microprocessor, a microcontroller, and/or digital signal processor (DSP), as would be appreciated to one skilled in the art.

The control unit 50 includes a light-emission control unit 51, a received-light-level acquisition unit 52, a determination processing unit 53, a biometric-information measurement unit 54, and a calibration unit 55. A battery 41 supplies power to the control unit 50. The battery 41 is connected to the finger-mounted device 10 via a power supply cable. The finger-mounted device 10 may contain the battery 41.

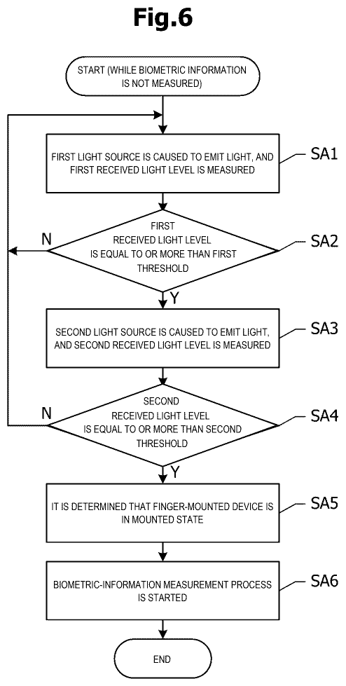

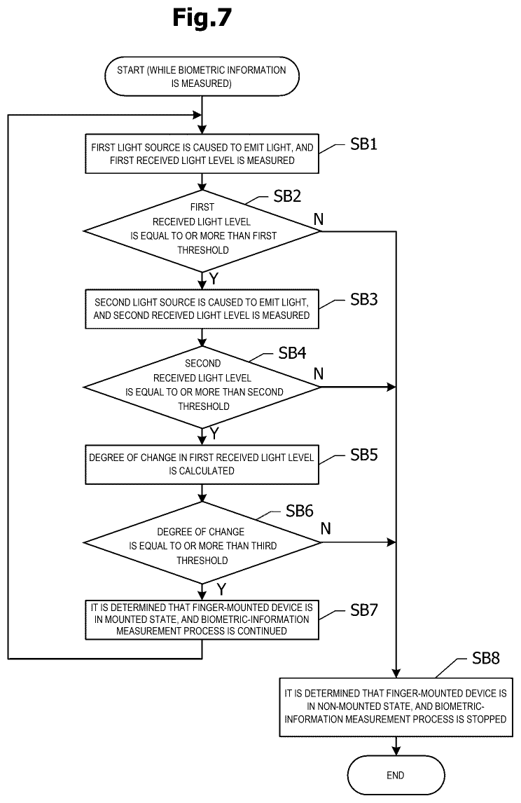

The light-emission control unit 51 controls light emission of the first light source 21 and the second light source 22. For example, the light-emission control unit 51 causes the first light source 21 and the second light source 22 to intermittently emit the light with different timings. The received-light-level acquisition unit 52 acquires a signal that represents the received light level of the light that is received by the light receiver 23. The determination processing unit 53 determines whether the finger-mounted device 10 is mounted on the finger. Processing that is performed by the determination processing unit 53 will be described in detail later with reference to FIG. 6 and FIG. 7.

The biometric-information measurement unit 54 performs a biometric-information measurement process in which biometric information such as a photoplethysmogram, a pulse, oxygen saturation, an autonomic nervous state, a respiratory state, a sleep state (such as identification of sleep and wakefulness, or the quality of sleep), blood pressure, a blood glucose level, blood flow, and/or vascular resistance is acquired based on the received light level of the light receiver 23 and a change in the received light level over time. The calibration unit 55 calibrates a threshold that is referred to when the determination processing unit 53 determines a mounted state. The processing of the calibration unit 55 will be described in detail later.

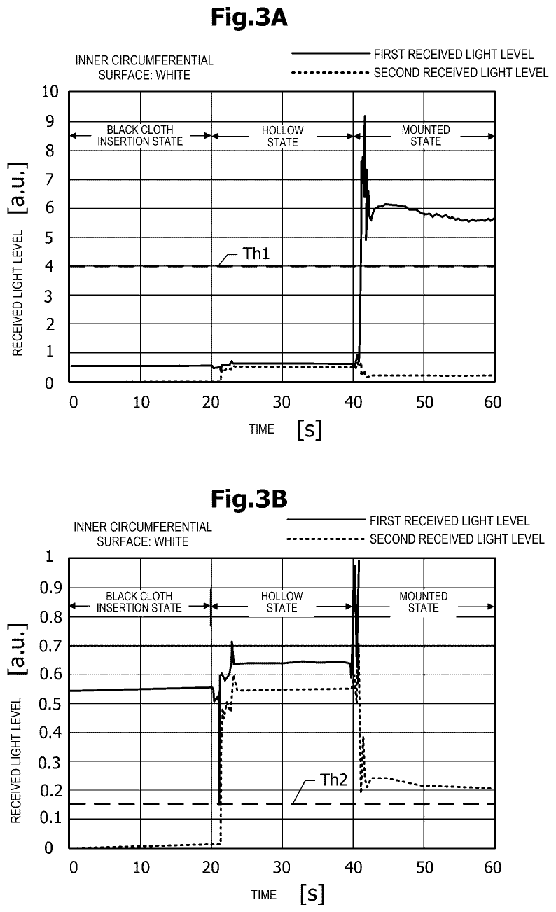

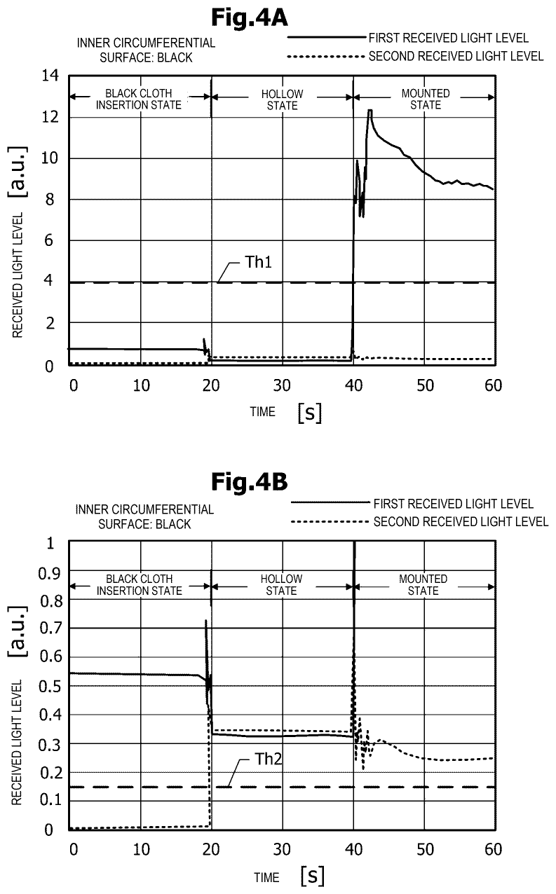

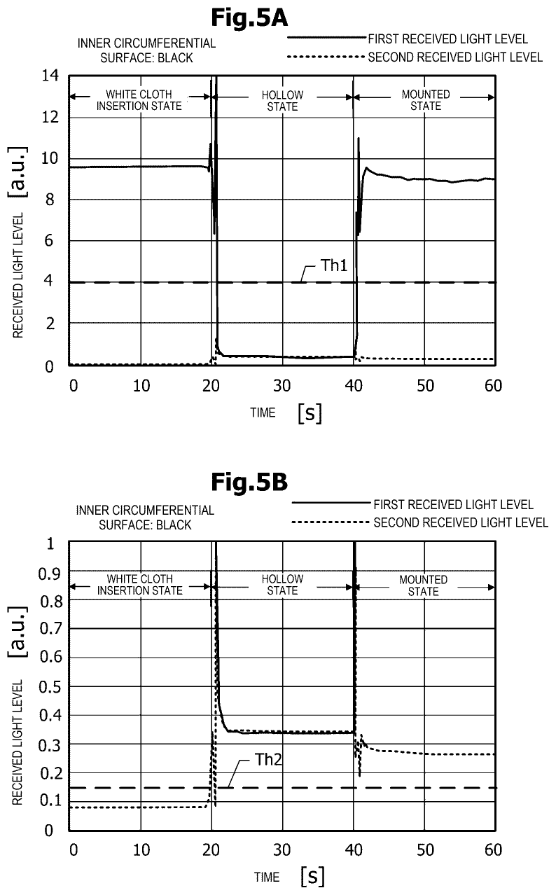

The change in the received light level measured by the light receiver 23 over time will now be described with reference to FIG. 3A to FIG. 5B. FIG. 3A to FIG. 5B are graphs illustrating the change in the received light level measured by the light receiver 23 over time. The horizontal axis of each graph represents time in seconds, and the vertical axis represents the received light level in arbitrary units. The graphs in FIG. 3B, FIG. 4B, and FIG. 5B are acquired by enlarging the vertical axes of the graphs in FIG. 3A, FIG. 4A, and FIG. 5A. Solid lines in the graphs represent the first received light level when the first light source 21 that emits the light in the wavelength range for green operates, and dashed lines represent the second received light level when the second light source 22 that emits the light in the wavelength range for near infrared operates.

FIG. 3A and FIG. 3B illustrate the received light level in the case where the inner circumferential surface of the finger-mounted device 10 is white. The graphs in FIG. 4A to FIG. 5B illustrate the received light level in the case where the inner circumferential surface of the finger-mounted device 10 is black.

The graphs in FIG. 3A to FIG. 4B illustrate the change in the received light level over time in a state in which black cloth is inserted into the finger-mounted device 10 (a black cloth insertion state), in a state in which nothing is inserted (a hollow state), and a state in which the finger is inserted (the mounted state). The graphs in FIG. 5A and FIG. 5B illustrate the change in the received light level over time in a state in which white cloth is inserted into the finger-mounted device 10 (a white cloth insertion state), the hollow state, and the mounted state.

As illustrated in FIG. 3A, FIG. 4A, and FIG. 5A, the first received light level in the mounted state is higher than the first received light level in the hollow state. For this reason, the measurement of the first received light level enables the hollow state and the mounted state to be identified. For example, comparison between the first received light level and a first threshold Th1 enables the hollow state and the mounted state to be identified. When the first threshold Th1 is set at “4” in arbitrary units on the vertical axes illustrated in FIG. 3A, FIG. 4A, and FIG. 5A, the hollow state and the mounted state can be identified with sufficiently high accuracy.

As illustrated in FIG. 5A, however, the first received light level in the mounted state is substantially equal to or slightly lower than the first received light level in the white cloth insertion state. The reason is that the intensity of scattered light due to the white cloth has the same degree as or higher than the intensity of the scattered light due to the finger. For this reason, the white cloth insertion state and the mounted state cannot be identified merely by measuring the first received light level.

As illustrated in FIG. 5B, the second received light level illustrated by the dashed line differs between the white cloth insertion state and the mounted state, and the second received light level in the mounted state is higher than the second received light level in the white cloth insertion state. In the graph illustrated in FIG. 5B, for example, comparison between the second received light level and a second threshold Th2 enables the white cloth insertion state and the mounted state to be identified. For example, in the case where the second threshold Th2 is set at “0.15”, and the second received light level is equal to or more than the second threshold Th2, the mounted state or the hollow state can be determined instead of the white cloth insertion state. Also in the graphs illustrated in FIG. 3B and FIG. 4B, the second received light level in the mounted state and the second received light level in the hollow state are equal to or more than the second threshold Th2.

From these findings, the finger-mounted device 10 can determine the mounted state instead of the hollow state, the black cloth insertion state, and the white cloth insertion state in the case where the first received light level is equal to or more than the first threshold Th1, and the second received light level is equal to or more than the second threshold Th2. FIG. 3A to FIG. 5B illustrate the case where the inner circumferential surface of the finger-mounted device 10 is white or black, and the inserted cloth is white or black. In the case of another color, however, the first received light level and the second received light level represent an intermediate color between black and white. Accordingly, even in the case where the color thereof is another color other than black and white, the mounted state can be determined.

A procedure for a mounted-state determination process that is performed by the control unit 50 (FIG. 2) while the biometric information is not measured will now be described with reference to FIG. 6. FIG. 6 is a flowchart illustrating the procedure for the mounted-state determination process that is performed by the control unit 50 of the finger-mounted device 10 according to the first exemplary embodiment while the biometric information is not measured.

The first light source 21 is first caused to emit the light, and the first received light level is measured (step SA1). Whether the first received light level is equal to or more than the first threshold Th1 is determined (step SA2). In the case where the first received light level is equal to or more than the first threshold Th1, the second light source 22 is caused to emit the light, and the second received light level is measured (step SA3). Whether the second received light level is equal to or more than the second threshold Th2 is determined (step SA4). In the case where the second received light level is equal to or more than the second threshold Th2, it is determined that the finger-mounted device 10 (FIG. 1) is in the mounted state (step SA5). The biometric-information measurement process is started (step SA6).

In the case where it is determined at step SA2 that the first received light level is less than the first threshold Th1, the procedure is repeated from step SA1. In the case where it is determined at step SA4 that the second received light level is less than the second threshold Th2, the procedure is repeated from step SA1.

At step SA6, the biometric-information measurement process is started, and the control unit 50 causes the first light source 21 and the second light source 22 to intermittently emit the light in turn and acquires the changes in the first received light level and the second received light level over time. Various kinds of biometric information can be acquired based on the changes over time. While the biometric information is measured, the first light source 21 and the second light source 22 emit the light at a frequency at which the waveform of a photoplethysmogram can be acquired, for example, a frequency of 100 Hz or more. It is not needed to increase the frequency more than necessary provided that the waveform of a photoplethysmogram can be acquired with sufficient accuracy. For example, the first light source 21 and the second light source 22 may emit the light at a frequency of 1000 Hz or less. In the case where high measurement accuracy is not needed, or in the case where oxygen saturation is measured, the frequency at which the first light source 21 and the second light source 22 emit the light may be reduced to about a frequency of no less than 50 Hz and no more than 100 Hz.

While the biometric information is not measured, it is not necessary to observe the waveform of a photoplethysmogram, and accordingly, it is not necessary to increase the frequency at which the first light source 21 and the second light source 22 emit the light. For example, the frequency at which the light is emitted when the mounted-state determination process is performed while the biometric information is not measured is no less than 0.1 Hz and no more than 50 Hz.

From the perspective of a reduction in power consumption, a pulse width when the first light source 21 and the second light source 22 emit the light is narrow. When the pulse width is too narrow, however, noise resistance decreases. For example, the pulse width is no less than 1 μs and no more than 100 μs.

the mounted-state determination process while the biometric information is measured will now be described with reference to FIG. 7. FIG. 7 is a flowchart illustrating a procedure for the mounted-state determination process that is performed by the control unit 50 (FIG. 1) while the biometric information is measured.

The control unit 50 first causes the first light source 21 to emit the light, and the first received light level is measured (step SB1). The first received light level and the first threshold Th1 are compared (step SB2). In the case where the first received light level is equal to or more than the first threshold Th1, the second light source 22 is caused to emit the light, and the second received light level is measured (step SB3). The second received light level and the second threshold Th2 are compared (step SB4). In the case where the second received light level is equal to or more than the second threshold Th2, the degree of the change in the first received light level over time is calculated (step SB5). For example, the amplitude of the waveform of the first received light level and the standard deviation of the first received light level are calculated.

While the biometric information is measured as illustrated in FIG. 7, the first light source 21 and the second light source 22 are caused to intermittently emit the light at a frequency higher than that while the biometric information is not measured as illustrated in FIG. 6. In this case, data for determining the mounted state as illustrated in FIG. 7 is collected in a manner in which time series data acquired at a high sampling frequency to measure the biometric information is decimated. For example, in the case where the sampling frequency for measuring the biometric information is 200 Hz, the number of the time series data is decimated to ⅛ for acquisition. In this case, the sampling frequency for mounting determination is 25 Hz.

The first received light level that is referred to when the degree of the change in the first received light level is calculated at step SB5 is measured at a high sampling frequency in the biometric-information measurement process. This enables the presence or absence of a photoplethysmogram to be detected.

The first threshold Th1 that is used at step SA2 (FIG. 6) for processing while the biometric information is not measured and the first threshold Th1 that is used at step SB2 (FIG. 7) for processing while the biometric information is measured are not necessarily equal to each other. Similarly, the second threshold Th2 that is used at step SA4 (FIG. 6) for the processing while the biometric information is not measured and the second threshold Th2 that is used at step SB4 (FIG. 7) for the processing while the biometric information is measured are not necessarily equal to each other.

In the case where the finger-mounted device 10 is in the mounted state, an amplitude or a standard deviation corresponding to the waveform of a photoplethysmogram is calculated as the value of the amplitude or the standard deviation. In the case where the finger-mounted device 10 is not mounted on the finger, no photoplethysmogram is detected, and accordingly, the amplitude of the waveform of the first received light level or the standard deviation of the first received light level is less than the value in the mounted state.

The degree of the change in the first received light level and a third threshold Th3 are compared (step SB6). In the case where the degree of the change in the first received light level is equal to or more than the third threshold Th3, it is determined that the finger-mounted device 10 is in the mounted state (step SB7), and the biometric-information measurement process is continued.

In the case where it is determined at step SB2 that the first received light level is equal to or less than the first threshold Th1, in the case where it is determined at step SB4 that the second received light level is equal to or less than the second threshold Th2, or in the case where it is determined at step SB6 that the degree of the change in the first received light level is equal to or less than the third threshold Th3, it is determined that the finger-mounted device 10 is in a non-mounted state, and the biometric-information measurement process is stopped (step SB8). After the biometric-information measurement process is stopped, the mounted-state determination process while the biometric information is not measured as illustrated in FIG. 6 is started.

The function of the calibration unit 55 (FIG. 2) of the control unit 50 will now be described.

The calibration unit 55 measures the first received light level and the second received light level with the finger-mounted device 10 unmounted from the finger and corrects the first threshold Th1 and the second threshold Th2, based on the measurement values thereof. For example, this processing is performed when the finger-mounted device 10 is completed and before product shipment. At this time, the first received light level and the second received light level are measured in a dark state in which no ambient light is present.

For example, the first received light level and the second received light level in the non-mounted state are measured for multiple finger-mounted devices 10 before product shipment. The first threshold Th1 of a finger-mounted device that has a large measurement value of the first received light level is set at a value greater than the first threshold Th1 of a finger-mounted device that has a small measurement value of the first received light level. Similarly, the second threshold Th2 of a finger-mounted device that has a large measurement value of the second received light level is set at a value greater than the second threshold Th2 of a finger-mounted device that has a small measurement value of the second received light level.

Technical effects according to the first exemplary embodiment will now be described.

According to the first exemplary embodiment, whether the finger-mounted device 10 is in the mounted state is determined by using the first received light level when the first light source 21 emits the light and the second received light level when the second light source 22 emits the light. For this reason, the accuracy of determination of whether the state is the mounted state is improved as described below.

For example, as illustrated in FIG. 3A and FIG. 4A, the magnitude of the first received light level greatly differs between the black cloth insertion state and the mounted state and between the hollow state and the mounted state. For this reason, the black cloth insertion state and the mounted state or the hollow state and the mounted state can be identified with high accuracy. As illustrated in FIG. 5A, it is difficult to identify the white cloth insertion state and the mounted state with accuracy merely by using the first received light level. According to the first exemplary embodiment, since the second received light level is also used, the white cloth insertion state and the mounted state can be identified with high accuracy as illustrated in FIG. 5B.

When the inner circumferential surface of the finger-mounted device 10 is black, as illustrated in FIG. 4B, a difference between the second received light level in the hollow state and the second received light level in the mounted state is small. For this reason, it is difficult to identify the hollow state and the mounted state with high accuracy merely by using the second received light level. According to the first exemplary embodiment, since the mounted state is determined also by using the first received light level, the hollow state and the mounted state can be identified with high accuracy.

According to the first exemplary embodiment, the frequencies at which the first light source 21 and the second light source 22 emit the light while the biometric information is not measured as illustrated in FIG. 6 are lower than the frequencies at which the first light source 21 and the second light source 22 emit the light while the biometric information is measured as illustrated in FIG. 7. For this reason, the power consumption while the biometric information is not measured is reduced.

According to the first exemplary embodiment, the first light source 21 and the second light source 22 emit the light with different timings, and consequently, the single light receiver 23 measures the first received light level when the first light source 21 emits the light and the second received light level when the second light source 22 emits the light. For this reason, the number of the light receiver 23 can be smaller than that of a structure that includes light receivers for respective light sources.

When a light source that emits light for measurement and a finger are not appropriately in close contact with each other, the quality of the waveform of a received light level decreases. According to the first exemplary embodiment, the first light source 21 and the second light source 22 are provided. Accordingly, when one of the light sources and the finger are not appropriately in close contact with each other, the other light source is operated, and the quality of the waveform of the received light level is high.

According to the first exemplary embodiment, the first light source 21 and the second light source 22 emit the light in different wavelength ranges, and accordingly, the biometric information suitable for the respective wavelengths can be acquired. For example, various component amounts and component concentrations in blood can be acquired by using an absorption spectrum as in a pulse oximeter. In addition, information about regions that have different depths can be acquired by using the fact that a penetration depth into a living body changes depending on the wavelength.

According to the first exemplary embodiment, as illustrated in FIG. 7, if the degree of the change in the first received light level while the biometric information is measured is less than the third threshold Th3, it is determined that the finger-mounted device 10 is in the non-mounted state, and the measurement of the biometric information is stopped (step SB8). In a method of determining the mounted state, based on only the magnitudes of the first received light level and the second received light level, in the case where the mounted state is mistakenly determined in spite of the non-mounted state, the biometric-information measurement process is continuously performed in the non-mounted state, and the power is consumed in vain. According to the first exemplary embodiment, in the case where the degree of the change in the first received light level is less than the third threshold Th3, in addition to the magnitudes of the first received light level and the second received light level, the biometric-information measurement process is stopped, and accordingly, unnecessary power consumption is reduced.

Technical effects exerted by the calibration unit 55 (FIG. 2) that has a function of correcting the first threshold Th1 and the second threshold Th2 will now be described. When a distance between the first light source 21 and the light receiver 23 is short, a part of the light emitted from the first light source 21 propagates in the resin member 25 while being reflected or scattered, and the amount of stray light incident on the light receiver 23 increases. The increase in the amount of the stray light increases the base level of the first received light level. In this case, the first threshold Th1 is increased depending on the increase in the base level.

The calibration unit 55 measures the first received light level with the finger-mounted device 10 unmounted from the finger. A received light level caused by the stray light is superimposed on the measurement value thereof. As illustrated in FIG. 3B and FIG. 4B, the first received light level does not significantly differ between the black cloth insertion state and the hollow state, and accordingly, it is thought that the majority of the first received light level in the hollow state is caused by the stray light.

According to the first exemplary embodiment, the first threshold Th1 is corrected based on the first received light level caused by the stray light, and accordingly, the first threshold Th1 is appropriately set for every device even in the case where the first received light level caused by the stray light varies among devices. Consequently, the accuracy of determination of the mounted state can be inhibited from being reduced. Similarly, the accuracy of determination of the mounted state can be inhibited from being reduced even in the case where the second received light level caused by the stray light varies among devices.

A color of the inner circumferential surface of the finger-mounted device 10 in some exemplary aspects will now be described.

When the first received light level and the first threshold Th1 are compared at step SA2 (FIG. 6), and when the second received light level and the second threshold Th2 are compared at step SA4 (FIG. 6), the received light level in the hollow state is low in order to identify the hollow state and the mounted state with high accuracy.

When the inner circumferential surface of the finger-mounted device 10 is white, as illustrated in FIG. 3B, the first received light level and the second received light level in the hollow state are in a range of no less than 0.55 and no more than 0.65. In contrast, when the inner circumferential surface of the finger-mounted device 10 is black, as illustrated in FIG. 4B and FIG. 5B, the first received light level and the second received light level in the hollow state are about 0.35. For this reason, the inner circumferential surface of the finger-mounted device 10 is black according to an exemplary aspect.

In particular, a region of the inner circumferential surface that reflects reflected light to be incident on the light receiver 23 is black in some examples. For example, when the annular structure 20 that is mounted on the finger is viewed in the insertion/removal direction (the z-direction) of the finger, a black portion is at a portion at which the inner circumferential surface of the annular structure 20 intersects with a perpendicular bisector that extends from the midpoint of a line segment both ends of which are located at the first light source 21 and the light receiver 23 toward the space that is surrounded by the annular structure 20.

The reflectance of the inner circumferential surface of the finger-mounted device 10 is 10% or less in the wavelength range in which the first light source 21 emits the light in an exemplary aspect. In the case where the reflectance of the inner circumferential surface is 10% or less in the wavelength range in which the first light source 21 emits the light, it can be said that the inner circumferential surface is black.

The shape of the annular structure 20 will now be described.

The finger-mounted device 10 is mounted on the finger and used, and accordingly, the annular structure 20 has a shape on which a sectional shape of the finger is reflected. For example, the inner circumferential surface of the annular structure 20 (FIG. 1B) has a shape that extends along the outer circumference of a circle or an ellipse when viewed in the insertion/removal direction (the z-direction) of the finger-mounted device 10. In the present specification, an “ellipse” does not necessarily mean a geometrically perfect ellipse. In the present specification, examples of the ellipse include a closed curve spaced apart from an outer circumferential line of the ellipse by a predetermined distance in a radial direction.

As for the sectional shape of the finger, an axis extending from the finger pad to the back of the finger is shorter than an axis extending from a side of the finger to the other side. A portion of the finger pad is richer in capillaries and has higher blood flow than a portion of the back, and accordingly, the portion of the finger pad is suitable for measuring the biometric information. For this reason, the finger-mounted device 10 is mounted such that the sensor module 30 (FIG. 1B) is in contact with the finger pad in some examples. The degree of close contact between the sensor module 30 and the portion of the finger pad can be increased in a manner in which the sensor module 30 is disposed at a portion that intersects with a minor axis of an xy section of the inner circumferential surface.

A finger-mounted device according to a modification to the first exemplary embodiment will now be described.

The brightness (the intensity of ambient light) around the finger-mounted device 10 is not necessarily constant. A part of the ambient light is incident on the light receiver 23 (FIG. 1B) while the biometric information is measured or while the biometric information is not measured, and accordingly, the first received light level and the second received light level change depending on a change in the intensity of the ambient light. For this reason, the accuracy of determination of the mounted state is reduced in some cases.

According to the present modification, the frequencies at which the first light source 21 and the second light source 22 (FIG. 1B) intermittently emit the light are sufficiently higher than the change rate of the intensity of the ambient light. A high pass filter is operated for the changes in the first received light level and the second received light level over time. For example, the cutoff frequency of the high pass filter is in a range of no less than 1 kHz and no more than 1 MHz. The high pass filter is operated, and consequently, the influence of the change in the intensity of soft ambient light is reduced.

### Second Exemplary Embodiment

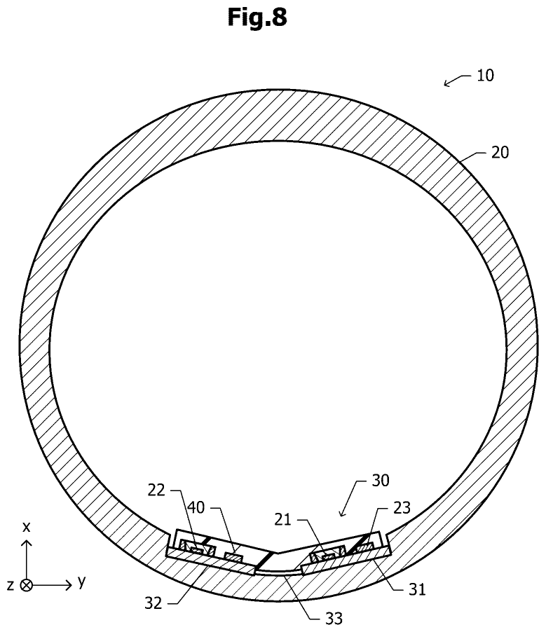

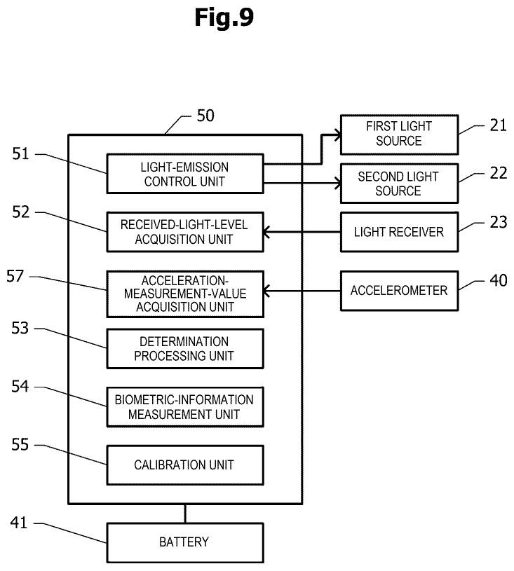

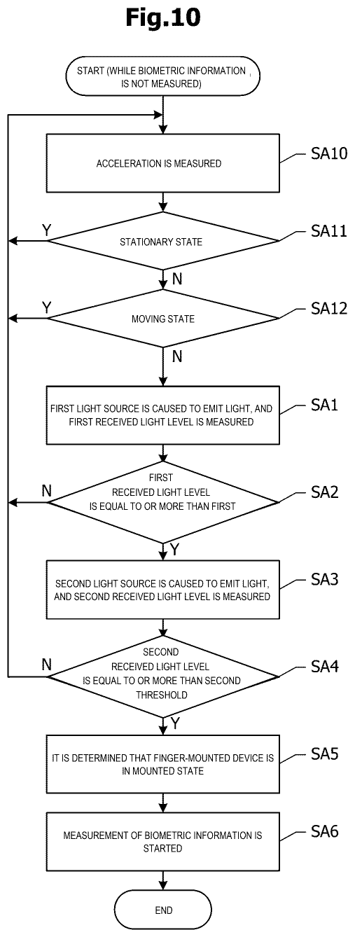

A finger-mounted device according to a second exemplary embodiment will now be described with reference to FIG. 8, FIG. 9, and FIG. 10. The description of components common to the finger-mounted device according to the first exemplary embodiment described with reference to FIG. 1A to FIG. 7 is omitted.

FIG. 8 illustrates a sectional view of a finger-mounted device 10 according to the second exemplary embodiment. The finger-mounted device 10 according to the second exemplary embodiment includes an accelerometer 40 in addition to the multiple components of the finger-mounted device 10 according to the first exemplary embodiment. For example, the accelerometer 40 is mounted on the rigid substrate 32 and measures acceleration in three directions perpendicular to each other.

FIG. 9 illustrates a block diagram for describing the function of the finger-mounted device 10 according to the second exemplary embodiment. The control unit 50 of the finger-mounted device 10 according to the second exemplary embodiment includes an acceleration-measurement-value acquisition unit 57 in addition to the multiple components of the control unit 50 of the finger-mounted device 10 according to the first exemplary embodiment. The acceleration-measurement-value acquisition unit 57 acquires the measurement values of the acceleration measured by the accelerometer 40. As for the finger-mounted device 10 according to the first exemplary embodiment, the determination processing unit 53 determines the mounted state, based on the first received light level and the second received light level. According to the second exemplary embodiment, however, the mounted state is determined based on the measurement values of the acceleration in addition to the first received light level and the second received light level.

FIG. 10 is a flowchart illustrating a procedure for the mounted-state determination process that is performed by the control unit 50 (FIG. 9) of the finger-mounted device 10 according to the second exemplary embodiment while the biometric information is not measured.

The acceleration-measurement-value acquisition unit 57 (FIG. 9) first acquires the measurement values of the acceleration from the accelerometer 40 (FIG. 9) (step SA10). For example, the measurement values of the acceleration may be acquired at a sampling frequency of no less than 10 Hz and no more than 30 Hz. Subsequently, the determination processing unit 53 (FIG. 9) determines whether the finger-mounted device 10 is in a stationary state, based on the measurement values of the acceleration (step SA11). In the case where the finger-mounted device 10 is not in the stationary state, whether a wearer of the finger-mounted device 10 is in a moving state is determined, based on the measurement values of the acceleration (step SA12). In the case where the finger-mounted device 10 is in the stationary state, or in the case where the wearer is in the moving state, the procedure is repeated from the acquisition of the measurement values of the acceleration (step SA10).

In the case where the wearer is not in the moving state, the control unit 50 of the finger-mounted device 10 according to the first exemplary embodiment performs the procedure from step SA1 to step SA6. That is, in the case where the finger-mounted device 10 is not in the stationary state, and the wearer is not in the moving state (referred to below as a non-moving state), the procedure is performed from step SA1 to step SA6. In the case where the first received light level is less than the first threshold Th1, or in the case where the second received light level is less than the second threshold Th2, the procedure is repeated from the acquisition of the measurement values of the acceleration (step SA10).

For example, in the case where all of the change rates of the measurement values of the acceleration in the three directions are equal to or less than a first acceleration-change-rate threshold, it is determined at step SA11 that the finger-mounted device 10 is in the stationary state. For example, in the case where at least one of the change rates of the measurement values of the acceleration in the three directions is equal to or more than a second acceleration-change-rate threshold, it is determined at step SA12 that the wearer is in the moving state. The second acceleration-change-rate threshold is greater than the first acceleration-change-rate threshold. The determination may be made based on the number of times the change rate of each measurement value of the acceleration exceeds the first acceleration-change-rate threshold within a predetermined time.

That is, in the case where at least one of the change rates of the measurement values of the acceleration in the three directions is more than the first acceleration-change-rate threshold, and all of the change rates of the measurement values of the acceleration in the three directions are less than the second acceleration-change-rate threshold, it is determined that the wearer is in the non-moving state.

Technical effects according to the second exemplary embodiment will now be described.

In the case where the finger-mounted device 10 is mounted on a user, a certain degree of acceleration is measured as a result of the motion of the user. According to the second exemplary embodiment, in the case where the finger-mounted device 10 is in the stationary state, the first light source 21 and the second light source 22 are not operated. For this reason, the power consumption in the mounted-state determination process while the biometric information is not measured is reduced.

In the case where the user moves with the finger-mounted device 10 contained in a pocket or a bag, the measurement values of the acceleration are more than the first acceleration threshold, and it is determined that the finger-mounted device 10 is not in the stationary state. If the mounted state is determined merely by using the measurement values of the acceleration, a state in which the finger-mounted device 10 is in a pocket or a bag and moves can be mistakenly determined as the mounted state. According to the second exemplary embodiment, determination is made based on the first received light level and the second received light level in addition to the measurement values of the acceleration, and consequently, the mounted state is unlikely to be mistakenly determined.

During walking or typing with the finger vigorously moved, the blood flow at a portion to be measured is disturbed, and accordingly, it is difficult to measure a photoplethysmogram with high accuracy. According to the second exemplary embodiment, in the case where it is determined at step SA12 that the wearer is in the moving state such as a walking or typing state, the biometric information is not measured. For this reason, the power consumption is reduced. The acceleration when the wearer performs various motions such that a photoplethysmogram is difficult to measure with high accuracy may be actually measured, and the second acceleration-change-rate threshold is set based on the measurement values thereof.

A finger-mounted device according to a modification to the second exemplary embodiment will now be described.

According to the second exemplary embodiment, the measurement values of the acceleration measured by the accelerometer 40 (FIG. 8 and FIG. 9) are used to determine whether the finger-mounted device 10 is in the mounted state. However, the motion of the wearer may be determined (gesture determination) by using the measurement values of the acceleration. In the case where the gesture determination is made, the sampling frequency at which the measurement values of the acceleration are acquired is higher than the sampling frequency that is used for the mounting determination and is no less than 20 Hz and no more than 300 Hz in some examples.

According to the second exemplary embodiment, the accelerometer 40 is mounted on the rigid substrate 32 (FIG. 8) on which the second light source 22 is mounted, but the accelerometer 40 may be mounted on another rigid substrate or another portion of the annular structure 20. A gyro-sensor may be used in addition to the accelerometer 40.

### Third Exemplary Embodiment

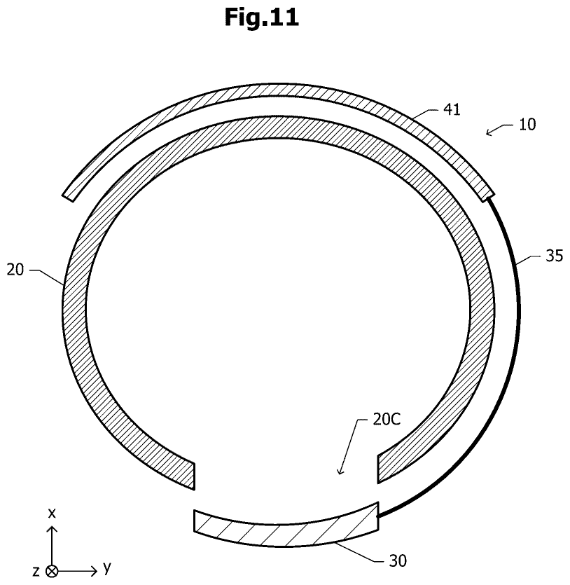

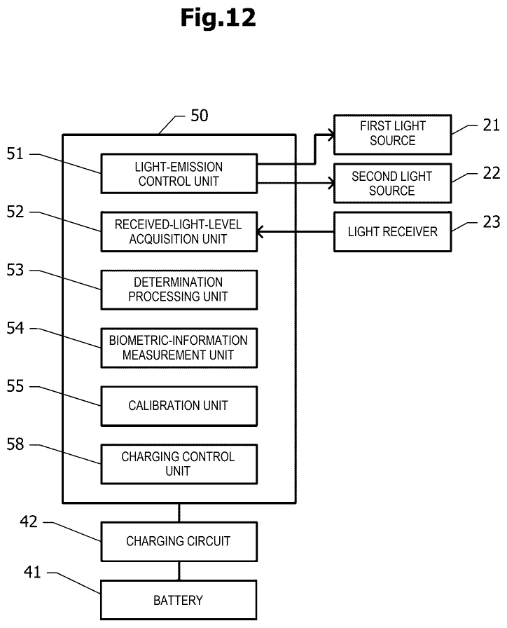

A finger-mounted device according to a third exemplary embodiment will now be described with reference to FIG. 11 and FIG. 12. The description of components common to the finger-mounted device according to the first exemplary embodiment described with reference to FIG. 1A to FIG. 7 is omitted.

FIG. 11 schematically illustrates a sectional view of a finger-mounted device 10 according to the third exemplary embodiment. According to the first exemplary embodiment (FIG. 1 and FIG. 2), the power is supplied from, for example, the battery 41 provided externally to the control unit 50 via the power supply cable. According to the third exemplary embodiment, however, the battery 41 is mounted on the annular structure 20 of the finger-mounted device 10.

The annular structure 20 has an opening portion 20C that extends from the inner circumferential surface thereof to the outer circumferential surface. The sensor module 30 is fitted into the opening portion 20C. The battery 41 is disposed along the outer circumferential surface of the annular structure 20. The battery 41 and the sensor module 30 are connected to each other with a cable 35 interposed therebetween. The battery 41 and the sensor module 30 may be connected to each other by a flexible substrate. In this case, a rigid-flexible substrate may be used as the rigid substrates 31 and 32 (FIG. 8) and the flexible substrate that are included in the sensor module 30 such that these are integrated into one piece.

In FIG. 11, the sensor module 30 is illustrated outside the opening portion 20C, but the sensor module 30 is fitted into the opening portion 20C and is fixed after assembling. Gaps are illustrated between the battery 41 and the outer circumferential surface of the annular structure 20 and between the cable 35 and the outer circumferential surface of the annular structure 20, but the battery 41 and the cable 35 are fixed to the outer circumferential surface of the annular structure 20 with an adhesive or adhesive tape after assembling.

FIG. 12 illustrates a block diagram for describing the function of the finger-mounted device 10 according to the third exemplary embodiment. The control unit 50 of the finger-mounted device 10 according to the third exemplary embodiment includes a charging control unit 58 in addition to the components of the control unit 50 (FIG. 2) according to the first exemplary embodiment. The finger-mounted device 10 according to the third exemplary embodiment further includes a charging circuit 42 that supplies charging power to the battery 41. The charging circuit 42 is controlled by the charging control unit 58, wirelessly receives power from the outside, and charges the battery 41.

The determination processing unit 53 receives information that represents a charging state from the charging control unit 58 and determines that the finger-mounted device 10 is in the non-mounted state when the battery 41 is in the charging state. When the battery 41 is in the charging state, the calibration unit 55 causes the first light source 21 and the second light source 22 to emit the light with different timings and corrects the first threshold Th1 that is used at step SA2 (FIG. 6) and step SB2 (FIG. 7) and the second threshold Th2 that is used at step SA4 (FIG. 6) and step SB4 (FIG. 7), based on the first received light level and the second received light level at this time.

Technical effects according to the third exemplary embodiment will now be described.

According to the third exemplary embodiment, the battery 41 is mounted on the annular structure 20, and accordingly, it is not necessary to connect a power supply cable to the finger-mounted device 10. For this reason, the wearer can perform various motions without being restricted by a power supply cable during mounting.

While the battery 41 is charged, the finger-mounted device 10 is removed from the finger and is placed on a wireless charger. While the battery 41 is charged, the calibration unit 55 corrects the first threshold Th1 and the second threshold Th2, and accordingly, the first threshold Th1 and the second threshold Th2 can be corrected in a state in which the finger-mounted device 10 is highly likely to be unmounted from the finger. If the first threshold Th1 and the second threshold Th2 are corrected whenever charging starts, the first threshold Th1 and the second threshold Th2 can be appropriately corrected depending on the amount of the stray light at this time even in the case where the amount of the stray light changes over time.

### Fourth Exemplary Embodiment

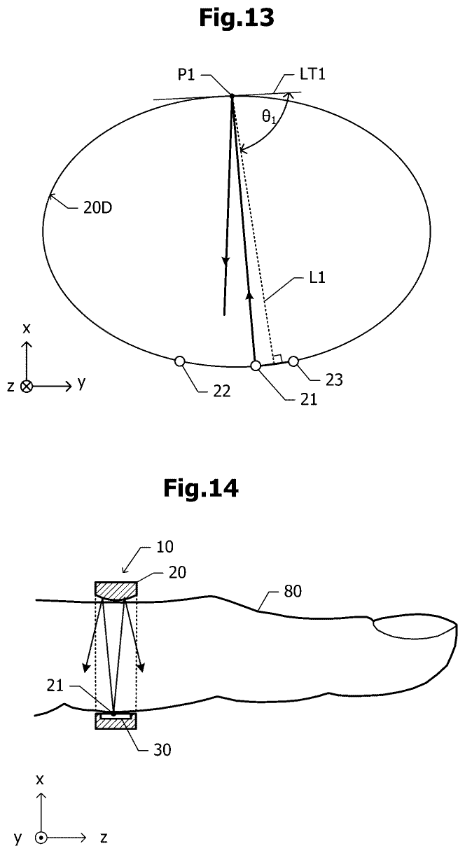

A finger-mounted device according to a fourth exemplary embodiment will now be described with reference to FIG. 13. The description of components common to the finger-mounted device according to the first exemplary embodiment described with reference to FIG. 1A to FIG. 7 is omitted.

FIG. 13 schematically illustrates an inner circumferential surface 20D of a finger-mounted device 10 according to the fourth exemplary embodiment when viewed in the insertion/removal direction (the z-direction) of the finger. A two-dimensional structure in an xy section will be described below.

The inner circumferential surface 20D has an elliptical shape on which the sectional shape of the finger is reflected. The longitudinal direction of the elliptical shape is defined as a y-direction, and the transverse direction thereof is defined as an x-direction. The first light source 21, the second light source 22, and the light receiver 23 are disposed near an end portion of a minor axis.

A portion at which the inner circumferential surface of the annular structure 20 intersects with a perpendicular bisector L1 that extends from the midpoint of a line segment both ends of which are located at the first light source 21 and the light receiver 23 toward the space that is surrounded by the annular structure 20 when viewed in the z-direction is designated by P1. The tangential direction of an xy section of the inner circumferential surface of the annular structure 20 at the intersection portion P1 is designated by LT1. An angle θ₁ formed between the perpendicular bisector L1 and the tangential direction LT1 at the intersection portion P1 differs from 90°. For example, the difference from 90° is 5° or more. That is, the angle formed between the perpendicular bisector L1 and the tangential direction LT1 at the intersection portion P1 is 85° or less. The condition of the angle θ₁ described above is likely to be satisfied when the first light source 21 and the light receiver 23 are arranged so as not to be symmetrical with respect to the minor axis and the major axis of the elliptical shape that is formed by the inner circumferential surface of the annular structure 20.

For example, the distance from the light receiver 23 to the second light source 22 is 5 mm or more.

Technical effects according to the fourth exemplary embodiment will now be described.

The angle θ₁ formed between the perpendicular bisector L1 and the tangential direction LT1 differs from 90°, and accordingly, reflected light after being emitted from the first light source 21 and specularly reflected at the intersection portion P1 is not incident on the light receiver 23. For this reason, the first received light level in the hollow state illustrated in FIG. 3A to FIG. 5B decreases. That is, the difference between the first received light level in the hollow state and the first received light level in the mounted state increases. As a result, the accuracy of determination of the mounted state is improved.

The inner circumferential surface 20D of the annular structure 20 at the intersection portion P1 is a mirror surface in order to sufficiently reduce the first received light level in the hollow state in some examples.

According to the fourth exemplary embodiment, when the distance from the light receiver 23 to the second light source 22 is 5 mm or more, the second received light level decreases in the case where a low-light-transmittance substance (such as black cloth or white cloth) is inserted into the finger-mounted device. In contrast, the second received light level increases in the case where a substance that is inserted into the finger-mounted device has a relatively high light transmittance in the wavelength range from red to near infrared such as a living body. The measurement of the second received light level enables the mounted state, the white cloth insertion state, or the black cloth insertion state to be identified with accuracy.

### Fifth Exemplary Embodiment

A finger-mounted device according to a fifth exemplary embodiment will now be described with reference to FIG. 14. The description of components common to the finger-mounted device according to the first exemplary embodiment described with reference to FIG. 1A to FIG. 7 is omitted.

FIG. 14 illustrates a sectional view of a finger-mounted device 10 according to the fifth exemplary embodiment with the finger-mounted device 10 mounted on a finger 80. In some examples, FIG. 14 illustrates a section that extends through the first light source 21 and extends through the center of the space that is surrounded by the inner circumferential surface of the annular structure 20. The sensor module 30 is in close contact with the finger pad of the finger 80. A section perpendicular to the circumferential direction of the annular structure 20 at a portion of the inner circumferential surface of the annular structure 20 that faces the first light source 21 convexly curves toward the space that is surrounded by the annular structure 20.

Light that is emitted from the first light source 21 and that is incident on the inner circumferential surface that faces the first light source 21 is reflected depending on the curved shape of the inner circumferential surface of the annular structure 20, and the majority of reflected light propagates in the z-direction so as to leave the annular structure 20. For this reason, the amount of the light incident on the light receiver 23 is smaller than that in the case where the inner circumferential surface is substantially flat. That is, the first received light level decreases.

Technical effects according to the fifth exemplary embodiment will now be described.

According to the fifth exemplary embodiment, the first received light level in the hollow state decreases, and accordingly, the difference between the first received light level in the mounted state and the first received light level in the hollow state increases in, for example, the graphs illustrated in FIG. 3A, FIG. 4A, and FIG. 5A. For this reason, the accuracy of determination of the mounted state is improved. The inner circumferential surface of the annular structure 20 that faces the first light source 21 is a mirror surface in order to improve the effect of decreasing the first received light level in the hollow state in some examples.

### Sixth Exemplary Embodiment

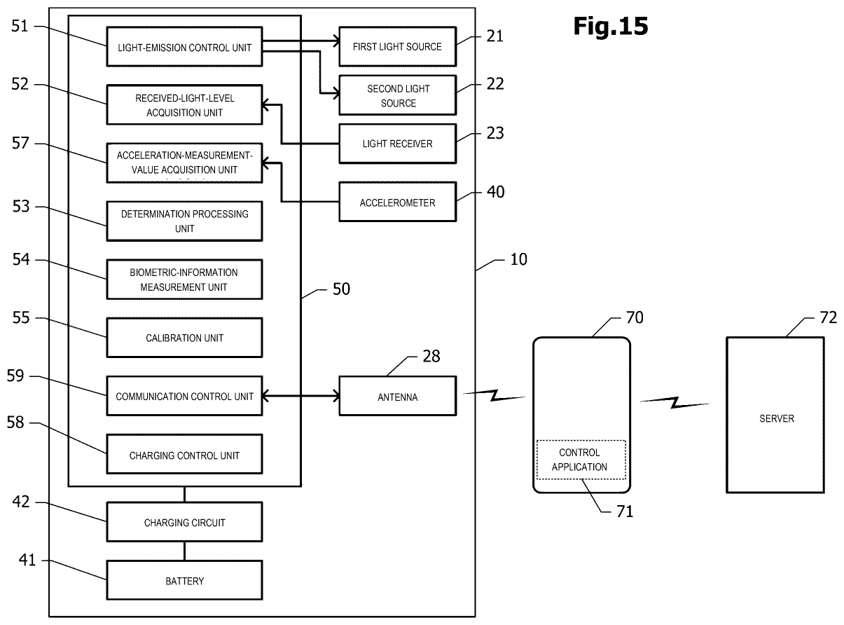

A finger-mounted device according to a sixth exemplary embodiment will now be described with reference to FIG. 15. The description of components common to the finger-mounted device according to the second exemplary embodiment described with reference to FIG. 8, FIG. 9, and FIG. 10 is omitted.

FIG. 15 illustrates a block diagram of the finger-mounted device 10 according to the sixth exemplary embodiment and an external device related to the operation of the finger-mounted device 10. The finger-mounted device 10 according to the sixth exemplary embodiment includes an antenna 28 and the charging circuit 42 in addition to the multiple components of the finger-mounted device 10 (FIG. 9) according to the second exemplary embodiment. The control unit 50 includes the charging control unit 58 and a communication control unit 59 in addition to the multiple components of the control unit 50 (FIG. 9) of the finger-mounted device 10 according to the second exemplary embodiment. The functions of the charging control unit 58 and the charging circuit 42 are the same as the functions of the charging control unit 58 and the charging circuit 42 according to the third exemplary embodiment (FIG. 12).

The control unit 50 exchanges data with the external device such as a control terminal 70 via the antenna 28. An example of the control terminal 70 is a smartphone. A control application 71 for controlling the finger-mounted device 10 is installed in control terminal 70. The user operates the control terminal 70, and consequently, various instructions are provided from the control terminal 70 to the finger-mounted device 10. Various kinds of information acquired by the finger-mounted device 10 are transmitted to the control terminal 70.

The control terminal 70 exchanges data with a server 72 via a network. The server 72 estimates the biometric information such as a pulse rate, blood pressure, a blood glucose level, blood flow, vascular resistance, a respiration number, an autonomic nerve state, and a sleep state, based on the various kinds of information received such as information about a pulse waveform.

Technical effects according to the sixth exemplary embodiment will now be described.

According to the sixth exemplary embodiment, the control terminal 70 provides a user interface. For this reason, a device for providing a user interface is not needed for the finger-mounted device 10. The finger-mounted device 10 and the control terminal 70 wirelessly exchange data with each other, and accordingly, it is not necessary to connect a communication cable to the finger-mounted device 10. Consequently, the motion and operation of the wearer are unlikely to be restricted while the biometric information is measured.

### Seventh Exemplary Embodiment

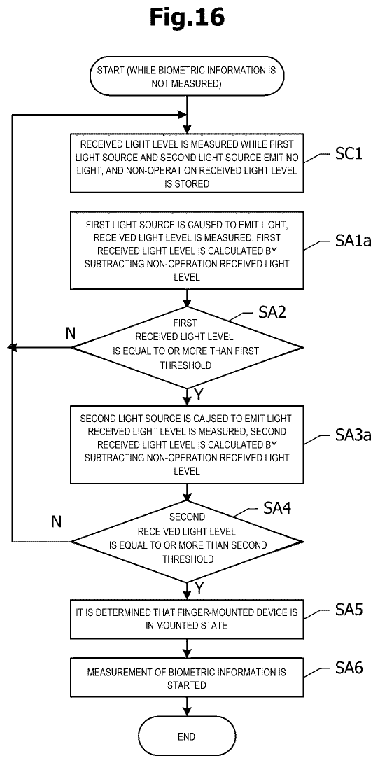

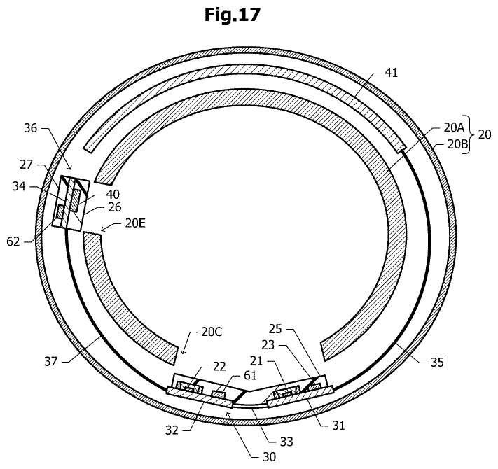

A finger-mounted device according to a seventh exemplary embodiment will now be described with reference to FIG. 16. The description of components common to the finger-mounted device according to the first exemplary embodiment described with reference to FIG. 1A to FIG. 7 is omitted.

FIG. 16 is a flowchart illustrating a procedure for a mounted-state determination process that is performed by the control unit 50 (FIG. 2) of a finger-mounted device 10 according to the seventh exemplary embodiment while the biometric information is not measured. According to the seventh exemplary embodiment, the received light level of the light receiver 23 (FIG. 1B and FIG. 2) is measured while the first light source 21 and the second light source 22 (FIG. 1B and FIG. 2) are not operated (step SC1), before the control unit 50 of the finger-mounted device 10 according to the first exemplary embodiment measures the first received light level at step SA1 illustrated in FIG. 6. The control unit 50 stores, as a non-operation received light level, the measurement value of the received light level. The non-operation received light level is caused by the ambient light such as lighting or sunlight.

The control unit 50 of the finger-mounted device 10 according to the seventh exemplary embodiment calculates the first received light level by subtracting the non-mounted received light level from the received light level when the first light source 21 emits the light (step SA1a), instead of step SA1 illustrated in FIG. 6. Similarly, the second received light level is calculated by subtracting the non-operation received light level from the received light level when the second light source 22 emits the light (step SA3a), instead of step SA3 illustrated in FIG. 6.

In the case where it is determined at step SA2 that the first received light level is less than the first threshold Th1, or in the case where it is determined at step SA4 that the second received light level is less than the second threshold Th2, the procedure is repeated from step SC1. The first threshold Th1 and the second threshold Th2 are set based on the first received light level and the second received light level with no ambient light incident on the light receiver 23 (FIG. 1B and FIG. 2).

In some cases, the intensity of the ambient light changes while the biometric information is not measured (FIG. 6). For example, in the case where the finger-mounted device 10 is mounted and is moved from a room to the outside, or in the case where the finger-mounted device 10 is placed on a table in a room, and lighting in the room is turned on, the intensity of the ambient light changes. According to the seventh exemplary embodiment, the non-operation received light level caused by the ambient light is measured, and the mounted state is determined in consideration of the non-operation received light level. For this reason, the influence of the change in the intensity of the ambient light is reduced, and the accuracy of determination of the mounted state is inhibited from being reduced.

A finger-mounted device according to a modification to the seventh exemplary embodiment will now be described.

According to the seventh exemplary embodiment, the non-operation received light level is considered in the mounted-state determination process that is performed while the biometric information is not measured. The mounted state may be determined in consideration of the non-operation received light level also while the biometric information is measured as described in FIG. 7. For example, the non-operation received light level is measured before step SB1 illustrated in FIG. 7.

When the non-operation received light level exceeds a reference level, the outputs of the first light source 21 and the second light source 22 are increased in some examples. This enables an S/N ratio to be improved. When the non-operation received light level further increases, a process of notifying the user of the fact that the ambient light is too strong is performed in some examples.

### Eighth Exemplary Embodiment

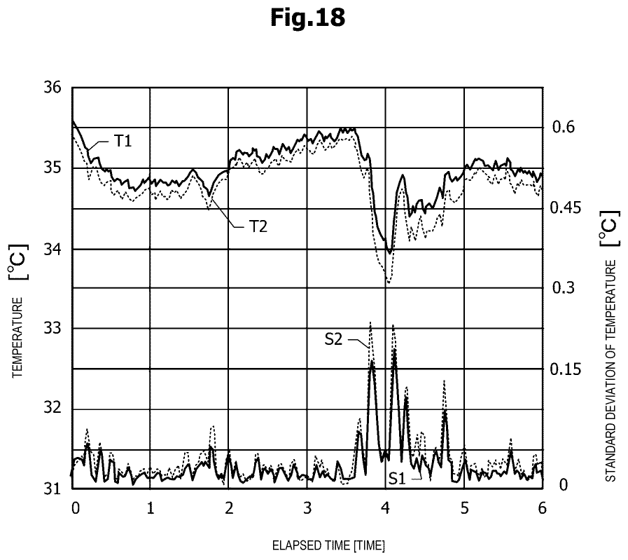

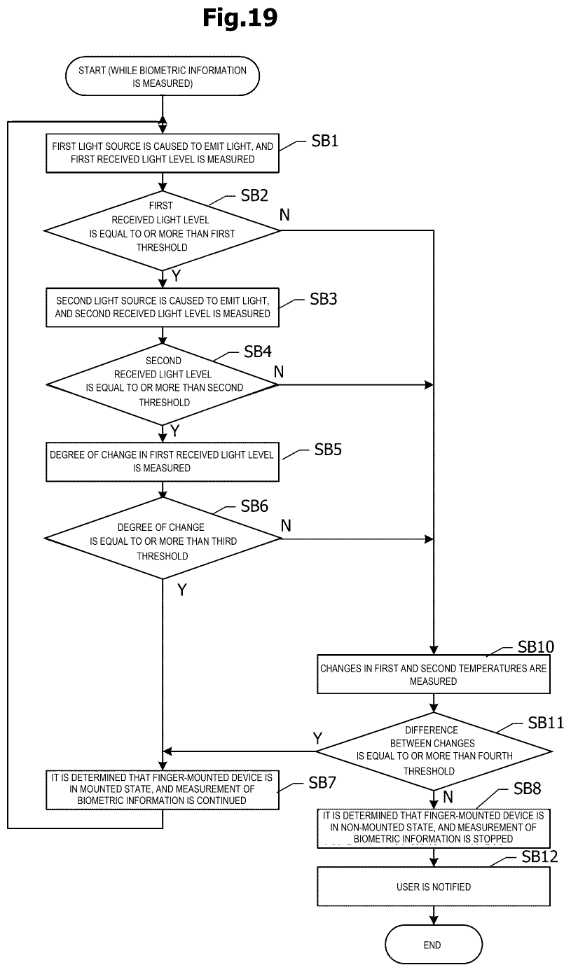

A finger-mounted device according to an eighth exemplary embodiment will now be described with reference to FIG. 17, FIG. 18, and FIG. 19. The description of components common to the finger-mounted device according to the third exemplary embodiment described with reference to FIG. 11 and FIG. 12 is omitted.

FIG. 17 schematically illustrates a sectional view of a finger-mounted device 10 according to the eighth exemplary embodiment. The annular structure 20 includes the inner member 20A and the outer member 20B. According to the third exemplary embodiment (FIG. 11), the inner member 20A has the single opening portion 20C. According to the eighth exemplary embodiment, however, the inner member 20A has two opening portions 20C and 20E. The sensor module 30 is fitted into the opening portion 20C, and a sensor module 36 is fitted into the opening portion 20E. In FIG. 17, the sensor modules 30 and 36 are illustrated outside the opening portions 20C and 20E. In practice, however, the sensor modules 30 and 36 are fitted into the respective opening portions 20C and 20E.

The sensor module 30 and the sensor module 36 are connected to each other by using a cable 37 that extends along the outer circumferential surface of the inner member 20A. In FIG. 17, gaps are illustrated between the cables 35 and 37 and the outer circumferential surface of the inner member 20A. In practice, however, cables 35 and 37 are in substantially close contact with the outer circumferential surface of the inner member 20A.

The sensor module 30 of the finger-mounted device 10 according to the eighth exemplary embodiment includes a first temperature sensor 61 in addition to the components of the sensor module 30 of the finger-mounted device 10 according to the first exemplary embodiment and the third exemplary embodiment. The first temperature sensor 61 is mounted on an inward facing surface of the rigid substrate 32. A surface of the first temperature sensor 61 that faces the space that is surrounded by the annular structure 20 is covered by the resin member 25. A thermistor or a sensor IC in which peripheral circuits are integrated can be used as the first temperature sensor 61.

The sensor module 36 includes a rigid substrate 34, the accelerometer 40, and a second temperature sensor 62. The accelerometer 40 is mounted on an inward facing surface of the rigid substrate 34, and the second temperature sensor 62 is mounted on an outward facing surface thereof. The accelerometer 40 is covered by a resin member 26, and the second temperature sensor 62 is covered by a resin member 27. A thermistor or a sensor IC in which peripheral circuits are integrated can be used as the second temperature sensor 62.

The outer member 20B faces the outer circumferential surface of the inner member 20A with the battery 41 and the cables 35 and 37 interposed therebetween. That is, the battery 41 and the cables 35 and 37 are disposed between the outer circumferential surface of the inner member 20A and the inner circumferential surface of the outer member 20B. Resin (not illustrated) is filled between the outer circumferential surface of the inner member 20A and the inner circumferential surface of the outer member 20B.

The first temperature sensor 61 and the second temperature sensor 62 are thus held by the annular structure 20. The first temperature sensor 61 is located at a position nearer than the outer circumferential surface of the annular structure 20 to the inner circumferential surface, and the second temperature sensor 62 is located at a position nearer than the inner circumferential surface of the annular structure 20 to the outer circumferential surface. The second temperature sensor 62 is thermally coupled with the outer member 20B with the resin member 27 interposed therebetween. The first temperature sensor 61 is thermally coupled with the finger with the resin member 25 interposed therebetween with the finger-mounted device 10 mounted on the finger.

For example, the outer member 20B is composed of metal or ceramics and has thermal conductivity higher than the thermal conductivity of the resin member 25. Examples of the metal used for the outer member 20B include stainless steel, titanium, tungsten, silver, gold, and platinum. The temperature of the finger is likely to be reflected on a first temperature that is measured by the first temperature sensor 61, and ambient temperature is likely to be reflected on a second temperature that is measured by the second temperature sensor 62.

FIG. 18 is a graph illustrating changes in temperatures that are measured by the first temperature sensor 61 and the second temperature sensor 62 over time and the standard deviations of the temperatures within a predetermined period of time. The horizontal axis represents an elapsed time in units of time, the left vertical axis represents the temperatures in ° C., and the right vertical axis represents the standard deviations of the temperatures in ° C.

A solid line T1 and a dashed line T2 in the graph represent the temperature (referred to below as the first temperature) that is measured by the first temperature sensor 61 and the temperature (referred to below as the second temperature) that is measured by the second temperature sensor 62. A solid line S1 and a dashed line S2 represent the standard deviations of the first temperature and the second temperature. A sampling interval for the temperatures is 30 seconds (the sampling frequency is 0.033 Hz). Each standard deviation is calculated by using a measurement value that is measured within 5 minutes.

The temperatures sharply drop when about 3.6 hours have elapsed because the wearer moves from a room to the outside where outside temperature is low. It can be seen that the first temperature is higher than the second temperature over the entire measurement time, and the standard deviation of the second temperature is higher than the standard deviation of the first temperature. That is, the degree of the change in the second temperature is higher than the degree of the change in the first temperature. When the sampling interval for the temperatures is shorter than 30 seconds, a difference between the standard deviations of the first temperature and the second temperature increases. For example, the sampling interval may be in a range of no less than 0.05 seconds and no more than 1 second.

The reason why the standard deviation of the second temperature is higher than the standard deviation of the first temperature will now be described. The second temperature sensor 62 is located near the outer circumferential surface of the annular structure 20, and the first temperature sensor 61 is located near the inner circumferential surface of the annular structure 20. For this reason, the second temperature is susceptible to a change in temperature in an external environment in the mounted state. In contrast, the first temperature is susceptible to the temperature of the finger of the wearer rather than the ambient temperature. The temperature of the finger is more stable than the ambient temperature, and accordingly, the standard deviation of the second temperature is higher than the standard deviation of the first temperature in the mounted state. In the non-mounted state, the first temperature and the second temperature are substantially equal to each other, and the standard deviations thereof are substantially equal to each other. A difference between the standard deviation of the second temperature and the standard deviation of the first temperature can be used as an indicator for determining whether the state is the mounted state.

The portion of the finger pad is richer in capillaries and has higher blood flow than the portion of the back and accordingly has higher skin temperature than the portion of the back and portions of the sides. The first temperature sensor 61 is, in some examples, thermally coupled with the portion of the finger pad rather than the portion of the back and the portions of the sides in order to measure the temperature of the finger by using the first temperature sensor 61. The first temperature sensor 61 is, in some examples, disposed near a portion of the inner circumferential surface of the annular structure 20 that intersects with the minor axis in order to thermally couple the first temperature sensor 61 with the portion of the finger pad. For example, the first temperature sensor 61 is, in some examples, disposed such that a distance from the position of intersection between the inner circumferential surface of the annular structure 20 and the minor axis is shorter than a distance from the position of intersection between the inner circumferential surface of the annular structure 20 and the major axis.

In particular, the second temperature sensor 62 is thermally coupled with the outer member 20B (FIG. 17) with the resin member 27 interposed therebetween. The outer member 20B has thermal conductivity higher than that of the resin member 25 that covers the first temperature sensor 61, and accordingly, the temperature of the outer member 20B is likely to change due to a change in outside temperature. For this reason, the second temperature that is measured by the second temperature sensor 62 that is thermally coupled with the outer member 20B is likely to change so as to follow the change in outside temperature.

For example, in the case where the outer member 20B comes into contact with a substance such as a hand, clothing, or water, in the case where the wearer moves to a location at a different outside temperature, in the case where the wind from an air conditioner hits the outer member 20B, or in the case where the direct sunlight hits the outer member 20B, the second temperature is likely to change. Even in this case, a change in the first temperature that is more susceptible to the temperature of the finger than the temperature of the outer member 20B is small.

In the case where the finger-mounted device 10 is in the non-mounted state, however, the first temperature is not affected by the temperature of the finger, the first temperature and the second temperature are accordingly substantially equal to each other, and the degrees of the changes in the first temperature and the second temperature are substantially equal to each other. For this reason, the degree of the change in the second temperature is higher than the degree of the change in the first temperature in the mounted state, and a difference between the degree of the change in the second temperature and the degree of the change in the first temperature is small in the non-mounted state. Accordingly, the mounted state can be determined by using the difference between the degree of the change in the first temperature and the degree of the change in the second temperature.

FIG. 19 is a flowchart illustrating a procedure for a mounted-state determination process that is performed by the control unit 50 (FIG. 2) of the finger-mounted device 10 according to the eighth exemplary embodiment while the biometric information is measured.

The procedure from step SB1 to step SB7 is the same as the procedure (FIG. 7) that is performed by the control unit 50 of the finger-mounted device 10 according to the first exemplary embodiment. According to the first exemplary embodiment, it is determined that the finger-mounted device 10 is in the non-mounted state, when any one of the following conditions is satisfied: the first received light level is less than the first threshold Th1; the second received light level is less than the second threshold Th2; and the degree of the change in the first received light level is less than the third threshold (step SB8 in FIG. 7).

According to the eighth exemplary embodiment, however, the degrees of the changes (for example, the standard deviations) in the first temperature and the second temperature are calculated (step SB10) when any one of the following conditions is satisfied: the first received light level is less than the first threshold Th1; the second received light level is less than the second threshold Th2; and the degree of the change in the first received light level is less than the third threshold. The first temperature and the second temperature are measured with a constant period.

Whether the difference between the degree of the change in the first temperature and the degree of the change in the second temperature is equal to or more than a fourth threshold Th4 is determined (step SB11). As described with reference to FIG. 18, the degree of the change in the second temperature is typically higher than the degree of the change in the first temperature.

In the case where the difference between the degree of the change in the first temperature and the degree of the change in the second temperature is equal to or more than the fourth threshold Th4, it is determined that the finger-mounted device 10 is in the mounted state, and the measurement of the biometric information is continued (step SB7). In the case where the difference between the degree of the change in the first temperature and the degree of the change in the second temperature is less than the fourth threshold Th4, it is determined that the finger-mounted device 10 is in the non-mounted state, and the measurement of the biometric information is stopped (step SB8). Subsequently, the user is notified of the fact that the measurement of the biometric information is stopped (step SB12). For example, the control terminal 70 illustrated in FIG. 15 is controlled, and a message is displayed on a screen that is displayed on the control terminal 70.

Technical effects according to the eighth exemplary embodiment will now be described.

According to the eighth exemplary embodiment, whether the state is the non-mounted state is determined also by using the first temperature and the second temperature in addition to the first received light level and the second received light level while the biometric information is measured. For this reason, the accuracy of determination is improved. In an example illustrated in FIG. 7, in the case where the result of determination at step SB2, SB4, or SB6 is “No” regardless of the mounted state, the non-mounted state is mistakenly determined, and the measurement of the biometric information is stopped. According to the eighth exemplary embodiment, such a mistaken determination is unlikely to occur.

For example, the calculation of the degree of a change in temperature requires about one minute. If temperature information is added for the determination process while the biometric information is not measured (FIG. 6), it takes about one minute until the measurement of the biometric information is started after the finger-mounted device 10 is mounted. The temperature information is, in some examples, excluded from the process of determining the mounted state while the biometric information is not measured in order to start the measurement of the biometric information right after mounting.

It goes without saying that the embodiments are described above by way of example, and the structures described according to the different embodiments can be partially replaced or combined. Like actions and effects of like structures according to the embodiments are not sequentially described for each embodiment. It is noted that the present disclosure is not limited to the embodiments described above. For example, a person skilled in the art can make various modifications, improvements, combinations, and others.

### REFERENCE SIGNS LIST

10 finger-mounted device
20 annular structure
20A inner member
20B outer member
20C opening portion
20D inner circumferential surface
20E opening portion
21 first light source
22 second light source
23 light receiver
25, 26, 27 resin member
28 antenna
30 sensor module
31, 32 rigid substrate
33 flexible substrate
34 rigid substrate
35 cable
36 sensor module
37 cable
40 accelerometer
41 battery
42 charging circuit
50 control unit
51 light-emission control unit
52 received-light-level acquisition unit
53 determination processing unit
54 biometric-information measurement unit
55 calibration unit
57 acceleration-measurement-value acquisition unit
58 charging control unit
59 communication control unit
61 first temperature sensor
62 second temperature sensor
70 control terminal
71 control application
72 server
80 finger

1. A finger-mounted device, comprising:
an annular structure configured to be mountable on a finger;
a first light source that is held by the annular structure and configured to emit first light into a space that is surrounded by the annular structure;
a second light source that is held by the annular structure and configured to emit second light having a different wavelength from the first light into the space that is surrounded by the annular structure;
a light receiver that is held by the annular structure and configured to receive light including reflected or scattered light associated with the first light and the second light; and
a control unit configured to:
control the first light source and the second light source to emit the first light and the second light intermittently; and
determine whether the annular structure is mounted on the finger based on a received light level that is measured by the light receiver,
wherein a distance from the light receiver to the second light source is longer than a distance from the light receiver to the first light source, and
wherein the control unit is configured to:
perform a mounted-state determination process that determines that the annular structure is mounted on the finger when a first received light level by the light receiver associated with the first light exceeds a first threshold, and a second received light level by the light receiver associated with the light exceeds a second threshold, and
perform a biometric-information measurement process that acquires biometric information based on at least the first received light level or the second received light level.

2. The finger-mounted device according to claim 1,
wherein the control unit is configured to start the biometric-information measurement process when the mounted-state determination process confirms that the annular structure is in a mounted state, and
wherein a first interval at which the first light source and the second light source emit the first light and the second light intermittently in the mounted-state determination process is longer than a second interval at which the first light source and the second light source emit the first light and the second light intermittently in the biometric-information measurement process.

3. The finger-mounted device according to claim 2, wherein the control unit is configured to continue the biometric-information measurement process when a change degree in the first received light level within a predetermined period of time is equal to or more than a third threshold and stop the biometric-information measurement process when the change degree is less than the third threshold.

4. The finger-mounted device according to claim 1, further comprising an accelerometer that is held by the annular structure, wherein the control unit is configured to:
determine that:
the annular structure is in a stationary state when a measurement value of acceleration by the accelerometer is of a first level,
the annular structure is in a moving state when the measurement value of the acceleration by the accelerometer is of a second level that is higher than the first level, or
the annular structure is in a non-moving state when the measurement value of the acceleration is of an intermediate level between the first level and the second level, and
perform the mounted-state determination process when the non-moving state is determined.

5. The finger-mounted device according to claim 1, wherein the control unit is configured to measure the first received light level and the second received light level with the annular structure being unmounted from the finger to obtain calibration measurement values and is configured to correct the first threshold and the second threshold according to the calibration measurement values of the first received light level and the second received light level.

6. The finger-mounted device according to claim 1, further comprising:
a battery configured to supply power to the control unit, the first light source, and the second light source; and
a charging circuit configured to supply charging power to the battery,
wherein the control unit is configured to:
control the charging circuit to charge the battery, and
while the battery is being charged, cause the first light source and the second light source to stop operating and determine that the annular structure is in a non-mounted state.

7. The finger-mounted device according to claim 1, wherein when viewed in an insertion/removal direction into the annular structure, the annular structure includes a black portion at an inner circumferential surface of the annular structure, the black portion intersecting with a perpendicular bisector that extends from a midpoint of a line segment ending at the first light source and the light receiver toward the space that is surrounded by the annular structure.

8. The finger-mounted device according to claim 1, wherein when viewed in an insertion/removal direction of the finger into the annular structure, an angle formed between a perpendicular bisector of a line segment that ends at the first light source and the light receiver and a tangential direction at an intersection portion between an inner circumferential surface of the annular structure and the perpendicular bisector is equal to or less than 85°.

9. The finger-mounted device according to claim 8, wherein when viewed in the insertion/removal direction of the finger into the annular structure, the annular structure extends along an ellipse.

10. The finger-mounted device according to claim 8, wherein the inner circumferential surface of the annular structure is a mirror surface.

11. The finger-mounted device according to claim 1, wherein the distance from the light receiver to the second light source is 5 mm or more.

12. The finger-mounted device according to claim 1, wherein a section perpendicular to a circumferential direction at a portion of an inner circumferential surface of the annular structure that faces the first light source convexly curves toward the space that is surrounded by the annular structure.

13. The finger-mounted device according to claim 1, wherein the first light that is emitted from the first light source has a wavelength range from blue to yellow-green.

14. The finger-mounted device according to claim 1, wherein the second light that is emitted from the second light source has a wavelength range from red to near infrared.

15. The finger-mounted device according to claim 1, further comprising:
an antenna that is held by the annular structure,
wherein the control unit is configured to exchange data with an external device via the antenna.

16. The finger-mounted device according to claim 1, wherein the control unit is configured to:
cause the light receiver to measure a non-operation received light level when the first light source and the second light source are controlled not to operate,
reduce the first received light level and the second received light level by the non-operation received light level to generate reduced received light levels, and
perform the mounted-state determination process, based on the reduced received light levels.

17. The finger-mounted device according to claim 1, further comprising a first temperature sensor that is held by the annular structure and is located closer to an inner circumferential surface than an outer circumferential surface of the annular structure.

18. The finger-mounted device according to claim 17, wherein a surface of the first temperature sensor that faces the space that is surrounded by the annular structure is covered by a resin member.

19. The finger-mounted device according to claim 17, further comprising a second temperature sensor that is held by the annular structure and is located closer to the outer circumferential surface than the inner circumferential surface of the annular structure.

20. The finger-mounted device according to claim 19, wherein a first thermal conductivity of a material of the outer circumferential surface of the annular structure is higher than a second thermal conductivity of a resin member that covers the first temperature sensor.
